(19) United States
(12) Patent Application Publication
(10) Pub. No.: US 2016/0054335 A1
(43) Pub. Date: Feb. 25, 2016

(54) MARKER FOR DIAGNOSING AGE-RELATED MACULAR DEGENERATION, AND METHOD FOR DIAGNOSING AGE-RELATED MACULAR DEGENERATION BY USING SAME

(71) Applicant: KOREA INSTITUTE OF SCIENCE AND TECHNOLOGY, Seoul (KR)

(72) Inventors: Cheolju LEE, Seoul (KR); Ji Eun LEE, Seoul (KR); Hye Jung KIM, Suwon-si (KR); Se Joon WOO, Seongnam-si (KR); Jeeyun AHN, Seoul (KR); Kyu Hyung PARK, Seongnam-si (KR); Eui Jin SUH, Goyang-si (KR)

(73) Assignee: SEOUL NATIONAL UNIVERSITY BUNDANG HOSPITAL, Seongnam-si, Gyeonggi-do (KR)

(21) Appl. No.: 14/779,898

(22) PCT Filed: Mar. 25, 2014
(86) PCT No.: PCT/KR2014/002523

§ 371 Date: Sep. 24, 2015

(30) Foreign Application Priority Data
Mar. 26, 2013 (KR) 10-2013-0032368

(51) Int. Cl. G01N 33/68; C07K 16/18; G01N 33/50

(52) U.S. Cl. CPC G01N 33/6887; G01N 33/5023; C07K 16/18; G01N 2333/4712; G01N 2800/7042

(57) ABSTRACT

A biomarker for diagnosing age-related macular degeneration includes a vinculin protein, and a diagnostic kit, a diagnostic method, and a method of screening a therapeutic material for age-related macular degeneration use the biomarker. The diagnostic kit and the diagnostic method of age-related macular degeneration are novel immunological diagnostic tools using the plasma of a patient, and have remarkable sensitivity that enables simple analysis of plasma without a biopsy, and thus can be useful for diagnosing age-related macular degeneration.

### TECHNICAL FIELD

The present invention relates to a marker for diagnosing age-related macular degeneration and a method of diagnosing age-related macular degeneration by using the same.

### BACKGROUND ART

Age-related macular degeneration (AMD) refers to a disease caused by various changes in retinal macula while aging. In western countries, AMD is a leading cause for typhlosis in the population aged 60 or over, and in South Korea, growing aged population may result in higher frequency thereof. Macula refers to the central part of a retina that is nervous tissue, and is populated with important cells responding to light-stimulation to be in charge of central visual acuity. When macula degenerates, visual disturbance may occur. This incidence is called macular degeneration. AMD is a degenerative disease that affects retinal pigment epithelium (RPE), Bruch's membrane, and choroid.

AMD is non-exudative (dry) or exudative (wet). In most cases, AMD is non-exudative. In the case of non-exudative AMD, lesions such as drusen or shrinking of retinal pigment epithelium occur in retina. In general, the non-exudative AMD does not cause serious typhlosis, but is likely to develop into its wet type. In the case of exudative AMD, choroidal neovascular vessels grow beneath the retina. These vessels may cause exudation or bleeding in the macula, affecting central vision, and between 2 months to 3 years after choroidal neovascularizations, causing typhlosis. Exudative AMD progresses very quickly so that within several weeks, visual acuity is highly likely to rapidly decrease. In the case of AMD, once a visual disorder develops, it is difficult to retain the original visual acuity. Accordingly, early diagnosis thereof is important. The early diagnosis may be performed through regular ophthalmic examinations. When AMD is suspected during ophthalmic examinations including funduscopic examination, ophthalmic close inspections including fluorescein angiography may be used for diagnosis. Examples of a nonsurgical treatment for AMD are photocoagulation, photodynamic therapy (PDT), transpupillary thermotherapy, and a drug treatment (for example, injection of a VEGF inhibitor). Examples of a surgical treatment for AMD are submacular surgery and macular translocation.

Vinculin is a protein that enables intercellular interaction associated with the attachment and mobility of cells. It is known that when vinculin decreases in concentration, intercellular connection becomes weak, and thus, cancer cells are highly likely to metastasize into other parts (Seimiya M et al., Hepatology 48:519-530, 2008). However, up until now, AMD-associated studies on a change in the expression level of the vinculin protein in blood have not been reported.

### TECHNICAL PROBLEM

An aspect provides a biomarker for diagnosing age-related macular degeneration (AMD), including a vinculin protein.

Another aspect provides a kit for diagnosing age-related macular degeneration, comprising an antibody that specifically binds to a vinculin protein or a fragment thereof.

Another aspect provides a method of diagnosing age-related macular degeneration or a method of screening a therapeutic material, the methods each including measuring an expression level of a vinculin protein.

### TECHNICAL SOLUTION

An aspect provides a biomarker for diagnosing age-related macular degeneration, including vinculin protein. The vinculin protein may have, for example, an amino acid sequence of SEQ ID NO: 1.

The vinculin protein may be a mammal intracellular cell membrane-skeletal protein having a molecular weight of about 123 kD, and has a sequence having about 20 to 30% identity to α-catenin and has a function similar thereto. The vinculin protein is detectable while being in plasma, and accordingly, may be used to diagnose age-related macular degeneration by using a simple method.

The vinculin protein shows a higher expression level in the blood, plasma, or serum of an individual having age-related macular degeneration than its normal control thereof. Accordingly, the development of age-related macular degeneration can be diagnosed by collecting the blood, plasma or serum of an individual who is suspected to have age-related macular degeneration and its normal control and comparing their expression levels of the vinculin protein. The individual may be a human being or a mammal other than a human being.

Another aspect provides a kit for diagnosing age-related macular degeneration, including an antibody that specifically binds to a vinculin protein or a fragment thereof. The vinculin protein may be, for example, amino acid sequence of SEQ ID NO: 1.

The fragment of the vinculin protein may be an immunogenic fragment. The immunogenic fragment has at least one epitope that is recognizable by an antibody against the vinculin protein. The antibody may be a polyclonal antibody or a monoclonal antibody. For example, the antibody may be a monoclonal antibody.

The polyclonal antibody may be prepared by using a method known to one of ordinary skill in the art. For example, the polyclonal antibody may be prepared by injecting the vinculin protein or the fragment thereof into a foreign host. An example of the foreign host may be a mammal, such as mice, rats, sheep, or rabbits. When the vinculin protein or the fragment thereof is administered by intramuscular injection, intraperitoneal injection, or subcutaneous injection, an adjuvant may be used together to increase antigenicity. Thereafter, from blood periodically collected from the foreign host, a serum having improved potency and showing specificity to an antigen may be collected, or an antibody may be separated or purified from the serum.

The monoclonal antibody may be prepared by using a method known to one of ordinary skill in the art. For example, the monoclonal antibody may be prepared by generating immortalized cell lines based on fusion. An example of the preparation method for the monoclonal antibody is as follows: vinculin protein is purified and an appropriate amount thereof is used to immunize a BALB/c mouse; or a polypeptide fragment of the vinculin protein is synthesized and combined with bovine serum albumin, and then, used to immunize a mouse, and then, antigen-production lymphocyte separated from the mouse is fused with human or mouse myeloma to generate immortalized hybridoma, and an enzyme-linked immunosorbent assay (ELISA) method is used to selectively proliferate only hybridoma cells that produce a target monoclonal antibody, and monoclonal antibodies are separated and purified from the culture. In some embodiments, the monoclonal antibody may be an commercially available antibody against vinculin protein.

The kit for diagnosing age-related macular degeneration may be prepared by using a method known to one of ordinary skill in the art. The kit may include, for example, a lyophilized antibody, a buffer, a stabilizer, or an inactivated protein. The antibody may be labeled by using radionuclides, fluorescors, enzymes, or the like.

The kit may be used for, for example, immunoassay. The immunoassay may be performed according to the protocol for existing immunoassay or immunostaining protocols. An immunoassay or immunostaining format may include radio immunoassay, radio immunoprecipitation, immunoprecipitation, ELISA, capture-ELISA, suppression or competition assay, sandwich assay, flow cytometry, immunofluorescence, immunoaffinity purification, or a combination thereof.

Another aspect provides a method of diagnosing age-related macular degeneration in a patient, and the method includes measuring an expression level of a vinculin protein in a biological sample obtained from an individual; and comparing the measured expression level of the vinculin protein with an expression level of vinculin protein in a control sample.

The individual may be a patient that is suspected of developing age-related macular degeneration. The individual may be a human being or a mammal other than a human being.

The expression level of vinculin protein may be measured by using an antibody that specifically binds to vinculin protein or a fragment thereof. The expression level may be measured by immunoassay or immunostaining. The expression level may be indirectly determined by measuring the mRNA amount of a gene that encodes vinculin protein. The measuring of the mRNA amount may be performed by using, for example, RT-PCR, competitive RT-PCR, qualitative RT-PCR, RNase protection assay, northern blot, or DNA chip.

The vinculin protein may have, for example, an amino acid sequence of SEQ ID NO: 1. The fragment of the vinculin protein and the antibody against the vinculin protein are the same as described above. The biological sample may be selected from blood, plasma, serum, an ophthalmic cell, ophthalmic tissue, and aqueous humor. For example, the biological sample may be blood, plasma, or serum, separated from a patient suspected of age-related macular degeneration.

When the method is performed by radio immunoassay, an antibody labeled with a radioactive isotope (for example, C14, I125, P32 and S35) may be used to detect the vinculin protein.

When the method is performed by ELISA, the method may be performed by (i) coating blood samples obtained from a normal individual and an individual suspected of age-related macular degeneration on the surface of a solid substrate; (ii) contacting as a primary antibody an antibody that specifically binds to the vinculin protein or the fragment thereof with the blood samples to induce an antigen-antibody reaction; and (iii) reacting the reaction product of the step (ii) with a secondary antibody bound to an enzyme; and (iv) detecting the activity of the enzyme. The solid substrate may be, for example, hydrocarbon polymer, glass, metal, gel, or microtiter plate. The hydrocarbon polymer may be, for example, polystylene or polypropylene.

The enzyme bound to the secondary antibody may be an enzyme that catalyzes a color reaction, a fluorescence reaction, a luminescence reaction, or an infrared ray reaction. The enzyme may be alkaline phosphatase, β-galactosidase, horseradish peroxidase, luciferase, or cytochrome P450. When the enzyme is alkaline phosphatase, a substrate for color reaction, such as bromochloroindolyl phosphate (BCIP), nitro blue tetrazolium (NBT), naphthol-AS-B1-phosphate, or enhanced chemifluorescence (ECF) may be used. When the enzyme is horseradish peroxidase, chloronaphthol, aminoethylcarbazole, diaminobenzidine, D-luciferin, lucigenin (bis-N-methylacryldinium nitrate), resorufin benzyl ether, luminol, amplex red reagent (10-acetyl-3,7-dihydroxyphenoxazin), p-phenylenediamine-HCl and pyrocatechol (HYR), tetramethylbenzidine (TMB), 2,2′-azine-di[3-ethylbenzthiazoline sulfonate] (ABTS), o-phenylenediamine (OPD), naphthol/pyronin, glucose oxidase, nitroblue tetrazolium (t-NBT), or phenzaine methosulfate (m-PMS) may be used as a substrate.

When the method is performed by capture-ELISA, the method may be performed by (i) coating as a capturing antibody an antibody that specifically binds to the vinculin protein or the fragment thereof on the surface of a solid substrate; (ii) contacting the capturing antibody with a blood sample obtained from an individual suspected of age-related macular degeneration to induce an antigen-antibody reaction; (iii) reacting the reaction product of the step (ii) with a detecting antibody which specifically reacts with the vinculin protein and to which a signal-generating label is bound; and (iv) detecting a signal generated by the label.

The detecting antibody may include a label generating a detectable signal. The label may be a chemical material (for example, biotin), an enzyme (alkaline phosphatase, β-galactosidase, horseradish peroxidase, and cytochrome P450), a radioactive material (for example, C14, I125, P32, and S35), a fluorescent material (for example, fluorescein), a luminescent material, a chemiluminescent material, a fluorophore used for fluorescence resonance energy transfer (FRET), or a combination thereof.

Regarding the ELISA method and the capture-ELISA method, the measurement of the enzyme's activity or the signal may be performed by using various methods known in the art. Due to the detection of the signal, the vinculin protein may be qualitatively or quantitatively assayed. For example, when biotin is used as a label, the signal may be detected using streptavidin, and when luciferase is used as a label, the signal may be detected using luciferin.

In some embodiments, the method may be performed by using a microchip and an automated microarray system. For example, an antibody that specifically binds to the vinculin protein or the fragment thereof is immobilized on a microchip, and then, the antibody is reacted with a biological sample separated from an individual suspected of age-related macular degeneration to detect the vinculin protein.

When an expression level of the vinculin protein in the biological sample obtained from an individual is higher than in its control sample, the increase in the expression level indicates that the individual has age-related macular degeneration. Accordingly, an individual having a higher expression level of the vinculin protein than that in its control may be diagnosed to have age-related macular degeneration. For example, when immunoassay results of a sample obtained from an individual suspected of age-related macular degeneration shows that a signal from the vinculin protein is stronger than that of a sample obtained from its normal control, the individual may be diagnosed as having age-related macular degeneration.

Another aspect provides a method of screening a material for treating age-related macular degeneration, wherein the method includes: contacting a cell containing a vinculin protein with a test material; and measuring an expression level of the vinculin protein in the cell to identify whether the expression is suppressed.

The cell containing the vinculin protein may be, for example, a retina pigment epithelial cell. Regarding the screening method, the term “test material” refers to an unknown material that is used for screening and is used to determine whether the test material affects the expression level of the vinculin protein. The test material may include, for example, a chemical material, a nucleotide, antisense-RNA, siRNA (small interference RNA), or a natural extract. The measuring of the vinculin protein or the expression levels of the vinculin protein is the same as described above. When the expression of the vinculin protein in the cell that has contacted the test material is suppressed, the test material may be determined to be a material for treating age-related macular degeneration.

### ADVANTAGEOUS EFFECT

A marker associated with age-related macular degeneration, a diagnostic kit including a material that is capable of detecting the age-related macular generation, and a diagnostic method using the same are novel immunological diagnostic tools using the plasma of a patient, and has remarkable sensitivity and enables simple analysis of the plasma without a biopsy, and thus can be useful for diagnosing age-related macular degeneration.

### DESCRIPTION OF DRAWINGS

FIG. 1 is a Venn diagram of the number of proteins obtained by profiling peptides obtained from plasma protein samples of 20 normal controls and 20 exudative AMD patients using a tandem mass spectrometer.

FIGS. 2A to 2D show mass spectra of peptide fragments derived from the vinculin protein.

FIG. 3 shows results of immunoblotting using anti-vinculin antibodies in plasma protein samples of a healthy control (HC), a non-exudative early AMD patient group (Early) and an exudative AMD patient group (Late). Prior to immunoblotting, each sample was subjected to electrophoresis.

FIGS. 4A and 4B show statistic results of the vinculin concentration measured by immunoblotting of FIG. 3. FIG. 4C shows a receiver operating characteristic (ROC) graph of the measured vinculin concentration.

FIG. 5 shows results of immunoblotting using anti-vinculin antibodis in new plasma protein samples of 60 healthy controls (HC), 10 non-exudative early AMD patients (Early), and 43 exudative AMD patients (Late). Prior to immunoblotting, each sample was subjected to electrophoresis.

FIGS. 6A and 6B show statistic results of the vinculin concentration measured by immunoblotting of FIG. 5. FIG. 6C shows a ROC graph of the measured vinculin concentration.

### MODE FOR INVENTION

Hereinafter, one or more exemplary examples will be described. However, these examples are presented herein for illustrative purpose only, and do not limit the scope of the present invention.

### EXAMPLE 1

### Identification of Vinculin in Plasma Protein of Age-Related Macular Degeneration (AMD) Patient

1-1. Preparation of Plasma Protein Sample of AMD Patient

Plasma samples were collected each in an amount of 40 μl from 20 exudative AMD patients and 20 healthy control participants. Each of the plasma samples was subjected to immunoaffinity chromatography using a MARS (multiple affinity removal system, Agilent) column. By tracking using an ultraviolet light absorption wavelength of 280 nm, a protein fraction that was not bound to the column was collected. The protein fraction was mostly free of 6 kinds of proteins which dominate a plasma protein: the six proteins are albumin, immunoglobulin A, immunoglobulin G, transferrin, trypsin inhibitor, and haptoglybin. The protein fraction was concentrated to prepare a plasma protein sample including only a protein having a size of 3 kDa or more.

1-2. Obtaining of Plasma Protein Fractions

Each of the 20 plasma protein samples of the patient group obtained in Example 1-1 was collected and mixed in the same amount, and each of the 20 plasma protein samples of the healthy group obtained in Example 1-1 was collected and mixed in the same amount. A protein amount in each of a mixed sample of the patient group and a mixed sample of the healthy group was adjusted to be 500 mg, and then, fractioning was performed thereon according to a molecular weight by using a gel-eluted liquid fractionation entrapment electrophoresis (GELFrEE 8100 fractionation system) system (Tran J C & Doucette AA(2008) Gel-eluted liquid fraction entrapment electrophoresis: an electrophoretic method for broad molecular weight range proteome separation. Analytical chemistry 80(5):1568-1573). Then, electrophoresis was performed for 2 hours using a gel column at 240 V. The gel column consisted of a 1 cm, 12% T stacking gel and a 3 cm, 4% T stacking gel. A time for a dye to be eluted by passing through a gel column was set as ‘0’ fraction, and after the ‘0’ fraction, 5 min fraction, 15 min fraction, 30 min fraction, 50 min fraction, and 120 min fraction were obtained. As a result, plasma protein fractions of the patient group and the healthy group, separated according to a molecular weight thereof, were obtained (patient group: 5 fractions, healthy group: 5 fractions).

1-3. Peptide Segmentation and Obtaining of Fractions

Dithiothreitol (DTT) was added to each of the plasma protein fractions obtained according to Example 1-2 until the concentration of DTT reached 10 mM, and reacted at 37° C. for 30 minutes to reduce disulfide bonds. Thereafter, for alkylation, iodoacetamide (IAA) was added thereto until the concentration of IAA reached 40 mM, and then the mixture reacted under a dark condition at room temperature for 1 hour, and the reaction product was diluted to 10 times using 50 mM NH₄HCO₃. Sequencing-grade trypsin (Promega) was added thereto in such a manner that a mass ratio of protein to trypsin was 40:1, and then, the mixture reacted at 37° C. for 16 hours. A trifluoroacetic acid (TFA) solution was added thereto until the concentration thereof reached 0.4%, thereby acidizing to a pH of 2.0 or less, and then, proteins were divided into peptide fragments. To identify more proteins, each fraction including peptide fragments was divided into 14 fractions according to an isoelectric point through OFFGEL fractionators (Agilent), and then a salt was removed therefrom by using a C18 SPE cartridge (Waters), followed by drying in a vacuum state.

1-4. Liquid Chromatography and Tandem Mass Spectrometry

The dry peptide fragments prepared according to Example 1-3 were dissolved in 10 μl of a 0.4% acetic acid solution, and then divided into portions each having an amount of 1 μl, and then, loaded into a reversed-phase MAGIC C18aq column (12 cm×75 μm, Eksigent MDLC system) mounted on a high-performance liquid chromatography (HPLC) apparatus coupled with an LTQ Obitrap mass spectrometer (Thermo Scientific). Before use, the column was adjusted to be in equilibrium by using a 95% buffer solution A (0.1% formic acid dissolved in water) and a 5% buffer solution B (0.1% formic acid dissolved in acetonitrile), and before the assay of samples, the column was repeatedly subjected to a concentration gradient process to be adapted to a concentration gradient condition. Each of the peptide samples was eluted at a constant speed for 80 minutes with a 10 to 30% buffer solution B in which a gradual concentration gradient occurred.

The mass spectrometer was used to scan a total mass range of 300 to 2000 m/z. When a total scan was performed on parent ions, which had been pre-examined, spectrums having sensibilities with 1^{st} to 6^{th} ranks from the top was identified, and the parent ions were captured to obtain the spectrum of daughter ions. Conditions used herein were a parent ion isolation width of 1.5 m/z, normalized collision energy of 25%, and a dynamic exclusion period of 180 seconds. When obtaining based on data continues, parent ions of which charge states did not match were removed. Data was obtained by Xcalibur software v2.0.7.

1-5. Data Analysis and Identification of Vinculin Protein

An MS/MS spectrum was screened by using SEQUEST (TurboSequest version 27, revision 12) with respect to the Human International Protein Index database (IPI, versions 3.44, European Bioinformatics Institute, http://www.ebi.ac.uk/IPI) having 72,065 protein entries, as well as contaminants. Conditions used herein were no enzyme, an MS/MS mass tolerance of 0.5 Da, and a mass tolerance of 15 ppm. For fixed modification, cysteine residue was set to 45.9877 Da, and for variable modification, to +15.9949 Da due to oxidation of methionine.

Identification and quantitative assay of peptide was performed by using Trans-Proteomic Pipeline (TPP, version 4.0, http://www.proteomecenter.org). A trypsin restriction and ‘monoisotopic masses’ options-added pepXML module was input to produce Sequest search results. For Peptide-Prophet, one having the probability of 0.05 or more, and for Protein-Prophet, one having the probability of 0.9 or more were elected as final data.

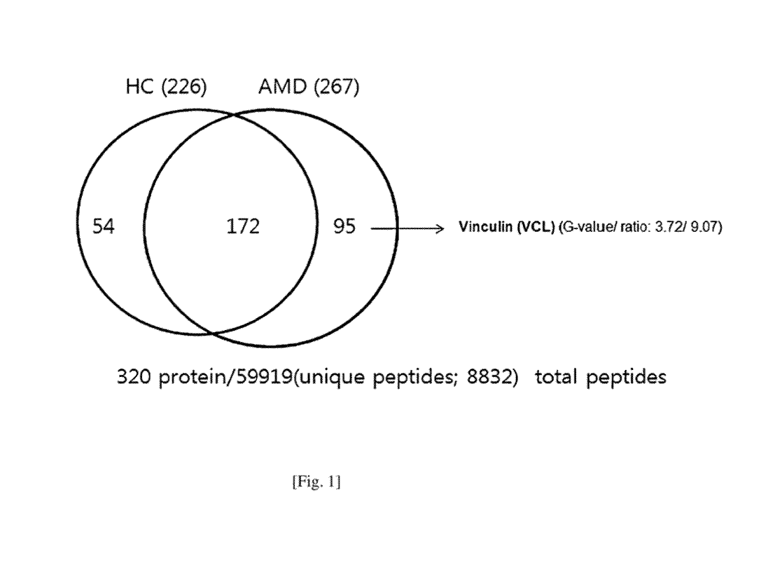

FIG. 1 is a Venn diagram of the number of proteins obtained by profiling peptides obtained from plasma protein samples of 20 normal controls and 20 exudative AMD patients using a tandem mass spectrometer. 8,832 unique peptides were identified from 320 proteins. By using spectral counting, the protein of the patient group was compared with the protein of the healthy group, and a protein that shows the expression level that is three times as high as that in the plasma of the patient group compared to the healthy group was classified as an exudative AMD patient-specific protein. g-value was calculated to confirm that the difference in the protein amount was significant.

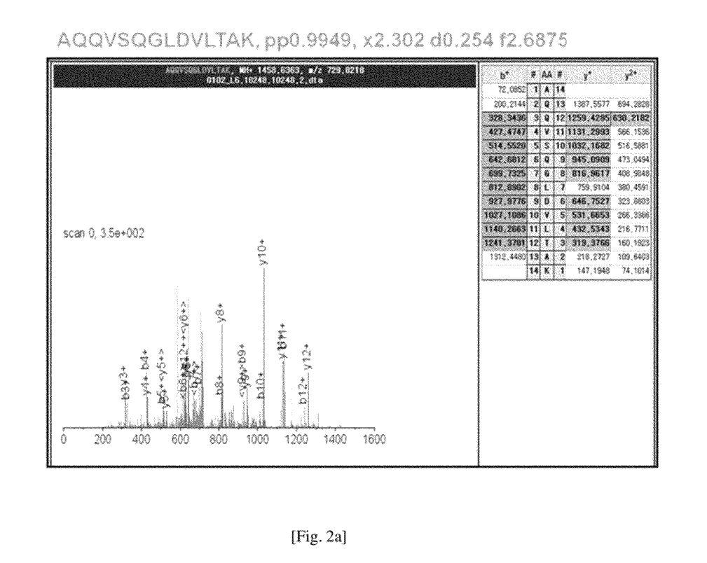

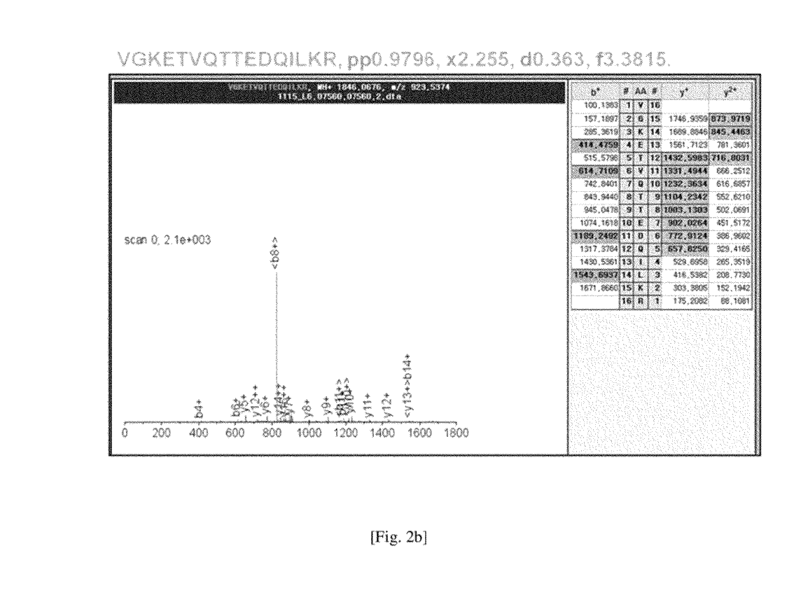

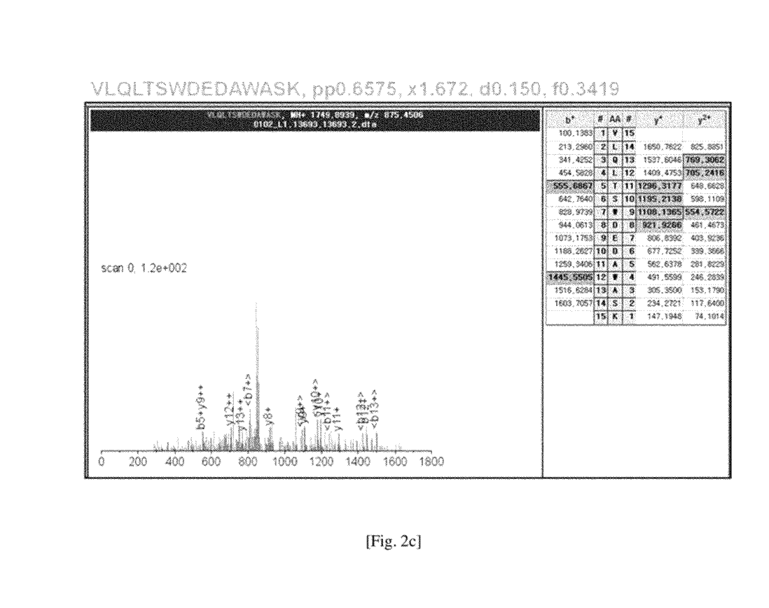

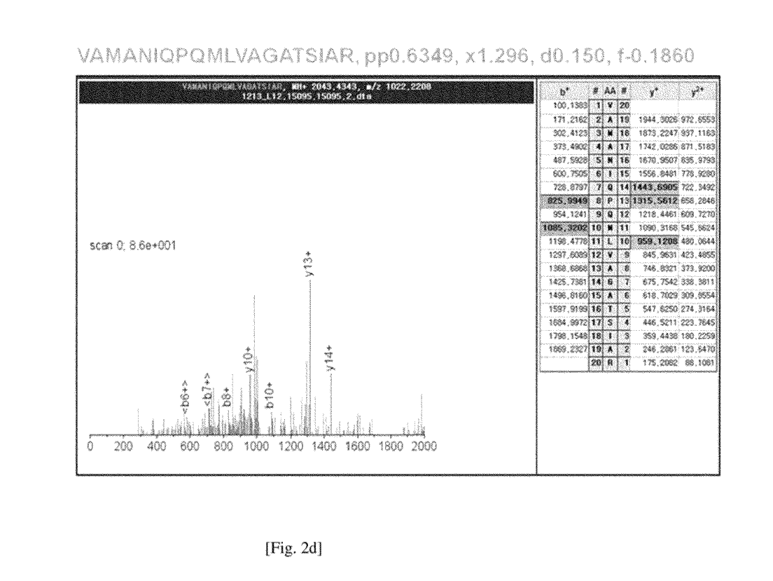

Assay results of the peptide identification show that a vinculin protein belonging to an exudative AMD patient-specific protein group was identified. FIGS. 2A to 2D show mass spectra of peptide fragments derived from the vinculin protein. From amino acid sequences of each peptide fragment, vinculin protein having an amino acid sequence of SEQ ID NO: 1 was identified.

### EXAMPLE 2

### Detection of Vinculin in AMD Patient Plasma by Using Immunoblotting

2-1. Preparation of Plasma Protein Sample of AMD Patient

Plasma samples were obtained from 22 non-exudative initial AMD patients, 58 exudative AMD patients, and 40 healthy controls. An amount of each of the obtained plasma samples was 40 μl. Each of the plasma samples was subjected to immunoaffinity chromatography using a MARS column. By tracking using an ultraviolet light absorption wavelength of 280 nm, a protein fraction that was not bound to the column was collected. The protein fraction was mostly free of 6 kinds of proteins which dominate plasma proteins: the six proteins are albumin, immunoglobulin A, immunoglobulin G, transferrin, trypsin inhibitor, and heptoglobin. The fraction was concentrated to prepare a plasma protein sample including only a protein having a size of 3 kDa or more.

2-2. Detection of Vinculin in Plasma by Immunoblotting

The plasma protein samples prepared according to Example 2-1 were diluted to 40 times by using PBS, and 10 μl of the diluted samples were mixed with 2% SDS-containing 1M Tris-HCL(pH 6.8) buffer solution, and then, electrophoresis was performed thereon by using 15% SDS polyacrylamide gel. A plasma protein separated by electrophoresis was transferred to a PVDF membrane in a buffer solution containing 50 mM Tris-HCl, 130 mM glycin, 0.05% SDS, and 12.5% methanol at a voltage of 90 V and at a current of 250 mA, and the transferred membrane was reacted for one hour in a 5% nonfat dried milk-containing TBST (Tris-buffered saline with Tween 20) buffer solution to which 0.02% sodium azide has been added. Thereafter, the membrane and anti-vinculin antibodies (ab18058, 1:100, Abcam) were reacted at 4° C. for 16 hours, and then, washed three times with TBST. Then, the membrane was reacted with mouse IgG-HRP(1:5000, GE Healthcare) which bind to anti-vinculin mouse immunoglobulin at 25° C. for 1 hour, and then, washed three times with TBST. Subsequently, the membrane was reacted with ECLplus (enhanced chemiluminescence, Amersham Bioscience), and then, was exposed to light on an X-ray film for 3 to 5 minutes to measure the concentration of protein.

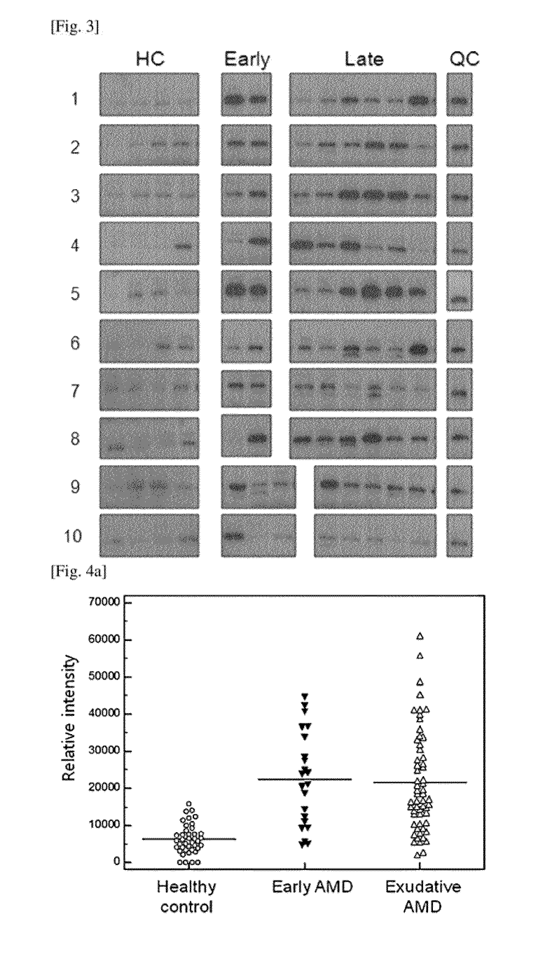

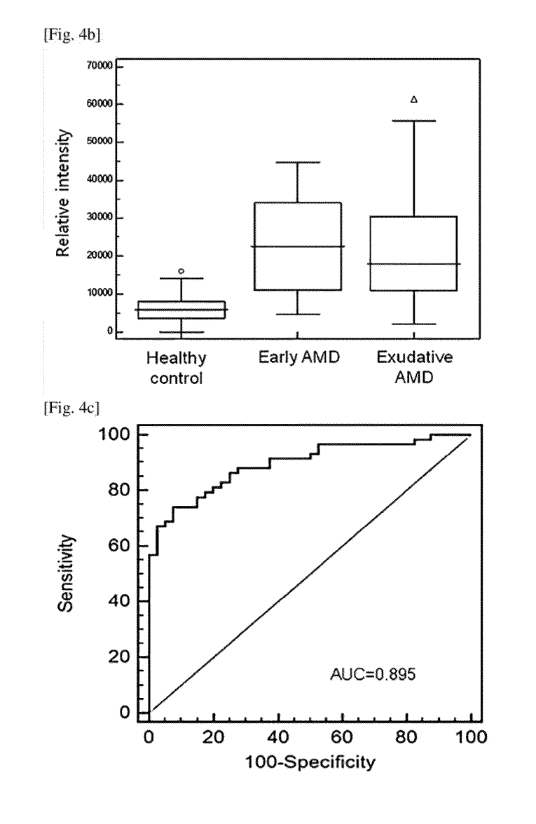

FIG. 3 shows results of immunoblotting using anti-vinculin antibodies in plasma protein samples of a healthy control (HC), a non-exudative early AMD patient group (Early) and an exudative AMD patient group (Late). Prior to immunoblotting, each sample was subjected to electrophoresis. The concentration of antigens identified by the immunoblotting was expressed as numeral values, which are shown in FIGS. 4A and 4B. It was confirmed that the concentration of vinculin in AMD patients was about 3.4 times as high as that in plasma of a healthy control.

FIGS. 4A and 4B show statistic results of the vinculin concentration measured by immunoblotting of FIG. 3. FIG. 4C shows a receiver operating characteristic (ROC) graph of the measured vinculin concentration. The ROC graph is a graph of all sensitivity/specificity pairs obtained by continuously changing a determination threshold in the entire range of obtained data, and usually indicates accuracy of tests (Zweig et al., Clin. Chem. 39: 561-577, 1993). The AUC (area under the ROC curve) of vinculin was 0.895 (SE 0.0312), which indicates that this assay has sensibility and accuracy.

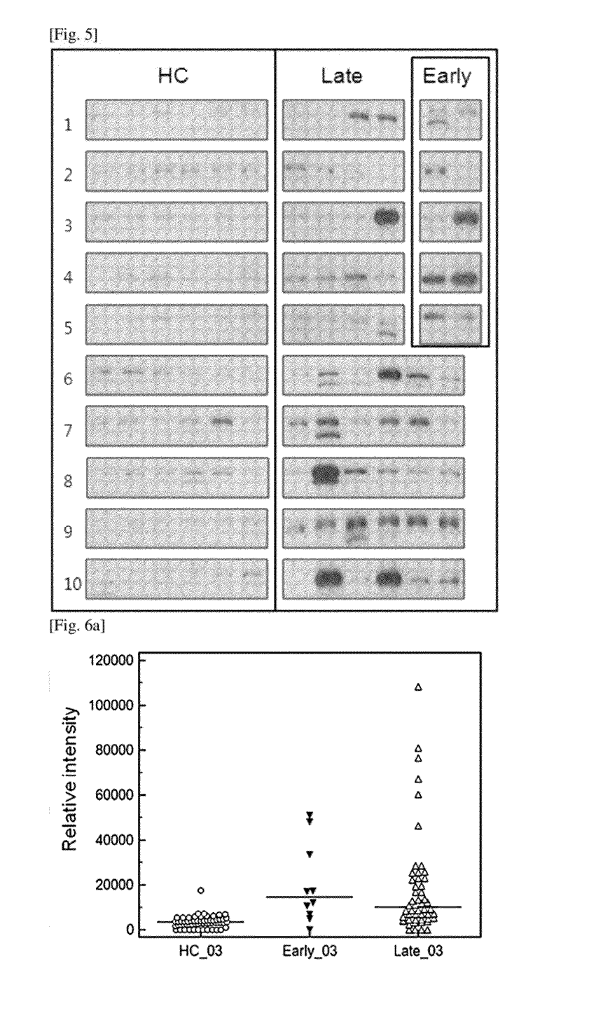

FIG. 5 shows results of immunoblotting using anti-vinculin antibodis in new plasma protein samples of 60 healthy controls (HC), 10 non-exudative early AMD patients (Early), and 43 exudative AMD patients (Late). Prior to immunoblotting, each sample was subjected to electrophoresis.

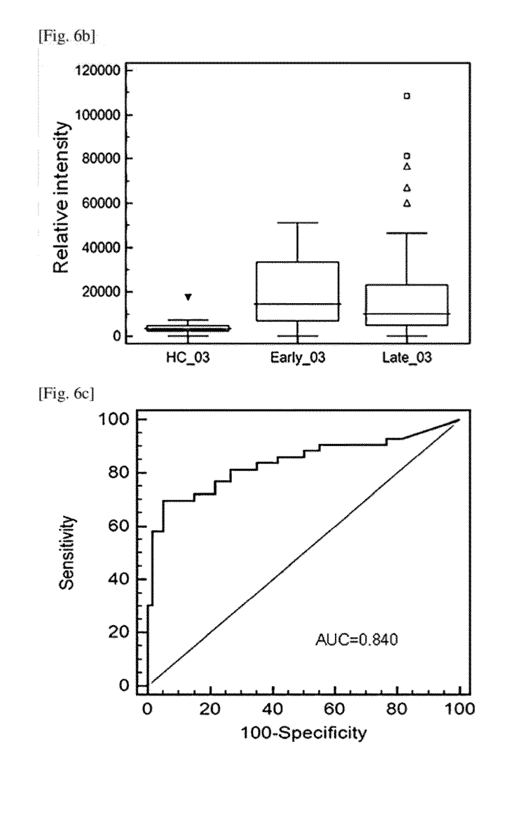

FIGS. 6A and 6B show statistic results of the vinculin concentration measured by immunoblotting of FIG. 5. FIG. 6C shows a ROC graph of the measured vinculin concentration. The AUC of vinculin was 0.838 (SE 0.0421), which indicates that this assay has sensibility and accuracy.

From these results, it was confirmed that by using an antibody that selectively recognizes a vinculin protein in plasma, a test group having a higher concentration of the vinculin protein than that in its healthy control can be diagnosed as an AMD patient.

1. A biomarker for diagnosing age-related macular degeneration, the biomarker comprising a vinculin protein.

2. The biomarker of claim 1, wherein the vinculin protein has an amino acid sequence of SEQ ID NO: 1.

3. A kit for diagnosing age-related macular degeneration, the kit comprising an antibody that specifically binds to a vinculin protein or a fragment thereof.

4. The kit of claim 3, wherein the vinculin protein has an amino acid sequence of SEQ ID NO: 1.

5. The kit of claim 3, wherein the antibody is a polyclonal antibody or a monoclonal antibody.

6. A method of diagnosing age-related macular degeneration in an individual, the method comprising:
measuring an expression level of a vinculin protein in a biological sample obtained from the individual; and
comparing the measured expression level of the vinculin protein with an expression level of a vinculin protein in a control sample.

7. The method of claim 6, wherein the expression level of the vinculin protein is measured by using an antibody that specifically binds to the vinculin protein or a fragment thereof.

8. The method of claim 6, wherein the vinculin protein has an amino acid sequence of SEQ ID NO: 1.

9. The method of claim 6, wherein the biological sample is selected from blood, plasma, serum, an ophthalmic cell, ophthalmic tissue, and aqueous humor.

10. The method of claim 7, wherein the antibody is a polyclonal antibody or a monoclonal antibody.

11. The method of claim 6, wherein an increase in the expression level of the vinculin protein in the biological sample obtained from the individual, compared to the expression level of the vinculin protein in the control sample, indicates that the individual has age-related macular degeneration.

12. A method of screening a material for treating age-related macular degeneration, the method comprising:
contacting a cell containing a vinculin protein with a test material; and
measuring an expression level of the vinculin protein in the cell to identify whether the expression is suppressed.

13. The method of claim 12, wherein the vinculin protein has an amino acid sequence of SEQ ID NO: 1.


SEQUENCE LISTING (1 sequence)

SEQ ID NO: 1 - protein, 1134 aa, Homo sapiens
MPVFHTRTIESILEPVAQQISHLVIMHEEGEVDGKAIPDLTAPVAAVQAAVSNLVRVGKETVQTTEDQILKRDMPPAFIKVENACTKLVQAAQMLQSDPYSVPARDYLIDGSRGILSGTSDLLLTFDEAEVRKIIRVCKGILEYLTVAEVVETMEDLVTYTKNLGPGMTKMAKMIDERQQELTHQEHRVMLVNSMNTVKELLPVLISAMKIFVTTKNSKNQGIEEALKNRNFTVEKMSAEINEIIRVLQLTSWDEDAWASKDTEAMKRALASIDSKLNQAKGWLRDPSASPGDAGEQAIRQILDEAGKVGELCAGKERREILGTCKMLGQMTDQVADLRARGQGSSPVAMQKAQQVSQGLDVLTAKVENAARKLEAMTNSKQSIAKKIDAAQNWLADPNGGPEGEEQIRGALAEARKIAELCDDPKERDDILRSLGEISALTSKLADLRRQGKGDSPEARALAKQVATALQNLQTKTNRAVANSRPAKAAVHLEGKIEQAQRWIDNPTVDDRGVGQAAIRGLVAEGHRLANVMMGPYRQDLLAKCDRVDQLTAQLADLAARGEGESPQARALASQLQDSLKDLKARMQEAMTQEVSDVFSDTTTPIKLLAVAATAPPDAPNREEVFDERAANFENHSGKLGATAEKAAAVGTANKSTVEGIQASVKTARELTPQVVSAARILLRNPGNQAAYEHFETMKNQWIDNVEKMTGLVDEAIDTKSLLDASEEAIKKDLDKCKVAMANIQPQMLVAGATSIARRANRILLVAKREVENSEDPKFREAVKAASDELSKTISPMVMDAKAVAGNISDPGLQKSFLDSGYRILGAVAKVREAFQPQEPDFPPPPPDLEQLRLTDELAPPKPPLPEGEVPPPRPPPPEEKDEEFPEQKAGEVINQPMMMAARQLHDEARKWSSKPGIPAAEVGIGVVAEADAADAAGFPVPPDMEDDYEPELLLMPSNQPVNQPILAAAQSLHREATKWSSKGNDIIAAAKRMALLMAEMSRLVRGGSGTKRALIQCAKDIAKASDEVTRLAKEVAKQCTDKRIRTNLLQVCERIPTISTQLKILSTVKATMLGRTNISDEESEQATEMLVHNAQNLMQSVKETVREAEAASIKIRTDAGFTLRWVRKTPWYQ